(19) United States
(12) Patent Application Publication
(10) Pub. No.: US 2009/0117163 A1
(43) Pub. Date: May 7, 2009

(54) Composition and Method for the Application of Human Synthetic Pheromones Using Substance Slowly Releasing Technological Bio Capsule in form of Drops in Clothing, Underwear, Jewels and Like

(75) Inventor: Alexandra Fede, Roma (IT)

Correspondence Address: HEDMAN & COSTIGAN P.C., 1185 AVENUE OF THE AMERICAS, NEW YORK, NY, 10036, US

(73) Assignee: Nute Partecipazioni S.p.A., Bologna (IT)

(21) Appl. No.: 12/225,472

(22) PCT Filed: Mar. 20, 2007
(86) PCT No.: PCT/IT2007/000201

§ 371 Date: Nov. 17, 2008

(30) Foreign Application Priority Data
Mar. 20, 2006 (IT) RM2006A000154

(51) Int. Cl. A61K 31/56; A61K 9/00; A61K 9/48; A61P 15/00

(52) U.S. Cl. 424/402; 514/170; 424/451; 424/452

(57) ABSTRACT

The invention regards a composition for the application of human synthetic pheromones in clothing, underwear, jewels, and like, comprising at least three positive human substances, e.g. androstenone, androstenediol and/or androsterone, said composition being predisposed in said clothing, underwear, jewel or like in such way to be diffused by the user by means of a simple action. The invention regards moreover a method for the application and dispensation of said composition, use of the same composition, to clothing, underwear, jewels and like incorporating the composition and a capsule as a carrier for the same composition.

The present invention regards a composition and method for the application of human synthetic pheromones in clothing, underwear, jewels, and like.

It is well known to all the scientists that various hormones are chemicals which, in the animals, result in a strong sexual desire.

It is further known that also man is affected, according to the same principle, although from different substances. However, said substances are not produced naturally by the human body in a such amount to stimulate the same type of response in the opposite sex.

The substances inducing above said effect in humans are long known and human synthetic pheromones have been synthesized.

The Applicant provided a composition and method for the application of human synthetic pheromones in clothing, underwear, accessories and like, in such a way to obtain the above-mentioned excitation effect in the opposite sex (or same sex) every time the subject desires so.

In other words, the composition according to the invention is put on as odorless composition, whereby, upon its release, the person on which the effect is desired to be obtained will recognize unconsciously the chemicals resulting in the sexual attraction and attention with sentimental interest.

Using the method according to the present invention, it is introduced a new BIO SIGNAS FASHION (emotional and sensitive) for clothing, underwear, jewels, etc with social and therapeutic values in comfortable fashionable context.

These and other results are obtained, according to the present invention, with the production of technologically up to dated micro containers or biocapsules, intimately hidden, containing an odorless composition that produces chemical benefits in a controlled way and satisfying the personal needs.

It is therefore a specific object of the present invention a composition for the application of human synthetic pheromones in clothing, underwear, jewels and like, comprising at least three/four positive human substances, e.g. androstenone, androstenediol and/or androsterone, said composition being predisposed in said clothing, underwear, jewel or like in such a way to be spread by the user through a simple action, for example by pressing.

According to the invention, said composition can comprise, in addition to said positive human substances, also additives, as urea, denatured alcohol or essential oils.

Preferably, according to the invention, a man designed composition can contain Androstenone in amount in a range from 0.017 to 0.027%, preferably 0.025%, Androstenediol in amount in a range from 0.011 to 0.018%, preferably 0.015%, Androsterone in amount in a range from 0.007 to 0.012%, preferably 0.011%, the balance being said additives.

Further according to the invention, a woman designed composition can contain Androstenone in amount in a range from 0.008 to 0.012%, preferably 0.010%, Androstenediol in amount in a range from 0.018 to 0.027%, preferably 0.025%, Androsterone in amount in a range from 0.013 to 0.020%, preferably 0.017%, the balance being said additives.

A particularly preferred embodiment of the composition, comprises adrostenone, preferably 0.4 mm, androstenolo, preferably 0.7 mm, androsterone, preferably 0.2 mm, androstadienone, preferably 0.1 mm, and copuline, preferably 0.1 mm, plus fragrances.

In addition the invention relates to a method for the application and dispensation of synthetic human pheromones in clothing, underwear, jewels and like comprising the predisposition of a composition comprising at least three positive human substances, particularly androstenone, androstenediol, androsterone and copulin (only for woman) in form of pressure and slowly releasing drop capsule, the positioning of at least one pressure capsule in clothing, underwear, jewel and like and in order to release the composition therein contained pressure must be exerted on said at least one capsule.

The invention moreover relates to the use of an as above defined composition in order to obtain the subliminal stimulation effect of vomeronasal organ (VNO), the only potential and considered organ for the unconscious recognition of chemicals determining sexual attraction.

The invention regards moreover clothing, underwear, jewel and like, comprising an as above defined composition.

Further the invention regards a seamless technologically up to dated capsule, wherein the above-mentioned composition is located, which assumes a perfect shape like a bright pearl, said capsule allowing distribution by dropping.

Particularly, the spreader is constituted of a concentric double spreader, wherein the internal spreader allows the content to be leaked and the external spreader supplies the inside enclosed heated composition.

The solution in object and particularly the specific way of fragrance positioning, which, as said, will practiced opportunely, allows an efficient distribution by targeting specific parts of the clothing correspondent to the points considered focal in the body.

Using the composition according to the invention, the user knowledge of the own visual identity by means of sensitive, psychological and medical well-being is enhanced.

In particular, the effects obtained with the composition according to the invention, are:
a chemical signal that influences specific examples of behavior from other members of the same species;
after the application, the effect of the substances for the women will be discovered to be prolonged on the man for another day;
more looks and smiles from the opposite sex (and potentially of the same sex);
the person will feel itself more attractive;
an unavoidable increase of the relationships;
a renovation of the current interpersonal relations;
a greater passion;
a greater feeling of relaxation and comfort;
an increased self-confidence;
a greater respect by the other sex;
a remarkable improvement of the working and business relations.

The chemicals move in the air, likely to smells, but act in completely different way.

Vomeronasal organ (VNO) is a small organ in the nose that recognizes chemicals; the effect of the chemicals is not influenced in any way from the experience, but it acts directly.

According to a scientific and technological definition they are substances acting like a molecular transmitter of information from a member to the other of the same species; or (Merrian-Webster), it is a substance which is produced by an animal and acts specifically as a stimulus in other individuals of the same species for one or more behavioral responses.

Scientific research proved that the chemicals are enough effective in attracting the persons of the same species. The airborne molecules influence various responses in the persons.

Some have feeling of well-being and confidence, others of sexual attraction.

Persons often producing high rate of these substances also in their body have a great success with members of the other sex.

A sub-set of androgens, i.e. adrenal androgens, comprising any 19-carbon-steroid synthesized from the adrenal cortex, the external portion of the adrenal gland, which act as weak steroids or steroid precursor, including DHEA, DHEA-S and adrostenedione, has been detected.

In addition to testosterone, other androgens include:
Dehydrosoandrosterone (DHEA): a steroid hormone produced from cholesterol in the adrenal cortex, which is the precursor of natural oestrogens. DHEA is also called dehydroisoandrosterone or deidroandrosterone.
Androstenedione (ANDRO): an androgenic steroid, that it is produced from the testis, cerebral cortex and ovary. While adrostenediones are metabolically converted in testosterone and other androgens, it represents the original structure of the oestrone. The use of androstenedione as integrator in athletic or body building has been prohibited by the Olympic International Commission, as well as by the other sport organizations.
Androstenediol: metabolite steroid acting as main regulator for the secretion of the gonadotropin.
Androsterone: it is a pheromone chemically produced during the degradation of androgens, or derived from progesterone, that exert lower virility effects, but with one seventh of testosterone intensity. It is found in the same amount in the plasma and urines of man and woman.
Dihydrotestosterone (DHT): it is a testosterone metabolite that is currently one of main androgens because it binds more strongly androgen receptors.
ADT is a steroid hormone with low androgenic activity. It is produced by the liver through the metabolism of the testosterone. It has been isolated firstly in 1931 by Adolf Friedrich Johann Butenandt and Kurt Tscherning, by distillation of more than 17000 liters of male urine recovering 50 milligrams of crystalline androsterone, enough to construct the chemical formula more similar to oestrone.

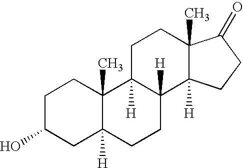

Urea (one of additives) is an organic compound consisting of carbon, nitrogen, oxygen and hydrogen with formula CON₂H₄ or (NH₂)₂CO. Also it is known as carbamide, especially according to rINN (controlled international denomination) used in Europe. For example, the medical compound hydroxy urea (old approved English name) is now hydroxycarbamide. Other names include carbammide resin, isourea, carbonil diamide and carbonil diamine.

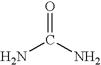

Copulins (pheromones) are secreted from the vagina and, although chemically different from other compounds considered pheromones, they act like the latter. Karl Grammer, a researcher from Vienna, showed pictures of women to two groups of men asking them to estimate the attraction of any woman. A group was exposed to copulins, while the other not. The copulin exposed group furnished higher score for all the women than not exposed one and more apparent the attraction of the woman, higher score of the copuline controlled group. Therefore the Grammer experiment suggests that the copulin is effective in order to attract a man.

Therefore, according to the invention, the used composition can comprise Androstenone, Androstenediol, Androsterone and Copulin.

In particular, the man designed composition can comprise 0.020% of Androstenone, 0.015% of Androstenediol and 0.010% of Androsterone, while for the woman designed composition 0.010% of Androstenone, 0.020% of Androstenediol, 0.017% of Androsterone, while copulins are fatty acid produced in the vagina, therefore only for the woman. Urea is a carbon organic compound.

An effective pheromone product can contain up to 99% of 5-Alfa-androst-16-e-3-one, in the highest effective solution percentage according to the invention and comprising Androstenone, Androsterone and Androstenediol.

Below chemicals suggested as human pheromones or synthetic equivalents thereof are shown.

Therefore, as already pointed out, Androstenone and Pheromonia are essential components in the composition according to the invention.

The airborne adrostanedione, a steroid produced from the odoriferous axillary glands, modifies the function of the limbic system and causes a positive affective reaction in the women, while the androstenedione reduces in a meaningful way the negative emotions in the men.

Abstract experiments aimed to find evidences of human pheromones focused on three partially overlapped possible classes of human pheromones: axillary steroids; aliphatic vaginal and vomeronasal organ stimulating acids. Examples of any class have been already patented in the past for commercial reasons and in some cases massively marketed.

Pheromones are defined as “substances that are secreted outside from every individual and received by another of the same species, resulting in a specific reaction in the latter—for example a particular behavior or a development process” (Karlson and Luscher 1959, p. 55). Generally two variants are recognized: release pheromones, which cause an immediate behavioral response and primary pheromones, which begin a long term psychological response.

Axillary steroids and other axillary odoriferous molecules

The most important armpit sweat odor results from aliphatic acids (Gower et al. 1988). I would emphasize that sweat odor can be eliminated using above said compounds when clothing from up-to-dated fiber systems are used. Moreover, ten steroids in the sweat of the human armpits were detected (Labows 1988). Three of these have been reported to be human pheromones: androstenone (5a-androst-16-en-one) androstenediol (5a-androst-16-en-3-a-ol) and androstanedione (androsta-4,16-dien-3-one). These steroids are released from the sweat of the armpits in too small amount to be odorous, but the axillary bacteria transform less odorous steroids (Gower et al. 1994), adding an “urine” and “musky” component to the axillary odor. Further large molecules, not easily identified, are present, adding to a such odor an acrid “burnt” component (Gower and Ruparelia 1993). One of these molecules is squalene, one of main members of pheromone fragrance of “tamarindi” (Saguinus fuscicollis), a New World primate (a Smith ET to 1985).

Human axillary steroids are produced from testis, ovary and adrenal glands and they do not seem to be by-products of the testosterone production (Gower and Ruparelia 1993). Young and middle age men produce higher levels of these steroids, like androstenone, than women, pre-pubescent and old men (Gower and Ruparelia 1993). It has been suggested that some of these steroids can be used as sexual male pheromones in the men, as it occurs in the pigs.

Axillary steroids and human behavior.

Various experiments proved that androstenol and androstenone are possible agents affecting affective and sexual reactions of people, especially young women. It is has been discovered that the odor of androstenol improves the feelings of the women versus men (Cowley et al. 1997; Cowley and Brooksbank 1991) and results in that ovulating women are “compliant” (Benton 1982) and androstenol attracts the women in a dark selection test.

Alfa-pheromone (N), i.e. “without friends” pheromone (friendless) (L), and “binding” pheromone (R) are sexual pheromones playing a fundamental role in the sex attraction. The mixture of three pheromones reflects a pheromone production by woman, wherein the “without friends” and “alpha” ones play the higher and smaller role, respectively.

The “without friends” pheromone is present at high concentrations on the woman skin and causes that the next persons become more sociable, friendly and loquacious.

Alfa-pheromone, present in lowest amounts on the woman skin, increases the respect and the gentility of the persons, and the man attention.

“Binding” pheromone is a sexual pheromone increasing the properties of the others two. Moreover it increases the man attention.

Moreover recently it has been discovered that copulins, produced only by women, have the property to attract men.

As far as the choice of textile and fibers, any type of fiber can be used, although man-made fibers are particularly favorable for the composition according to the invention.

The solution according to the invention borders on the synaesthesia, by mixing the molecule distribution within (VNO).

As above pointed out, according to a preferred embodiment of the present invention, the composition is introduced in a seamless capsule, which assumes a perfect shape like a bright pearl. Interface activity of the capsule performs the unique dropping technology. The diffuser is constituted of a concentric double spreader, wherein the internal spreader allows the content to be leaked and the external spreader supplies the inside enclosed heated composition.

By encapsulating the liquid it is possible to transform the liquids in solid particles or powder, whereby it is possible to use substances in the cases in which it would be difficult, if not impossible, to use liquids.

The better measurability and portability of this encapsulated liquid results in an easy combination with other substances. Thus it is also possible to improve considerably the delivery time of the substances, which would be exposed to oxidation when air exposed, modifying the quality or composition thereof. This allows also substances as aromas, easily evaporating, to boil at low temperature in order to be stored for long periods. This characteristic is used also to prevent the oxidation of the DHA and betacarotene.

It is possible moreover to control easily the release of encapsulated substances, depending on use thereof. Capsules like this allow an easy dissolution thereof, which easily are dissolved during a month. It is a capsule able to protect its content also at various temperatures and with programmed release during the time in order to extend the effect of the encapsulated substances.

The size of the capsule can be specified without limitation in order to meet all the demands.

The capsules are extremely homogenous, without variation of size and weight.

The thin shell allows the capsule to include substances normally contained therein in 50% higher amount, assuring the quality thereof.

The shell is constituted of a water soluble polymer like gelatin or agar.

The hardness of the capsule can be easily controlled, by modification of the material, water content and thickness.

For about 4-8 hours from the application time and by correct use of the composition according to the invention the desired results are obtained.

The present invention has been described by an illustrative, but not limitative way according to preferred embodiments thereof, but it is to be understood that variations and/or modifications will be apparent to the skilled in the art without departing from the scope thereof as defined in the appended claims.

1. Composition for the application of human synthetic pheromones in clothing, underwear, jewels and like, characterized in that it comprises at least three positive human substances, e.g. androstenone, androstenediol and/or androsterone, said composition being predisposed in said clothing, underwear, jewel or like in such way to be spread by the user through a simple action, as by pressing and like.

2. Composition according to claim 1 characterized in that said composition comprises, in addition to said positive human substances, also additives.

3. Composition according to claim 2 characterized in that said additives are constituted of urea and/or denatured alcohol and/or essential oils.

4. Composition as defined in claim 1 characterized in that in a man designed composition are present androstenone in amount in a range from 0.017 to 0.027%, preferably 0.025%, androstenediol in amount in a range from 0.011 to 0.018%, preferably 0.015%, androsterone or copulin in amount in a range from 0.007 to 0.012%, preferably 0.011%, the balance being said additives.

5. Composition according to 1 characterized in that in a woman designed composition are present androstenone in amount in a range from 0.008 to 0.012%, preferably 0.010%, androstenediol in amount in a range from 0.018 to 0.027%, preferably 0.025%, androsterone in amount in a range from 0.013 to 0.020%, preferably 0.017%, the balance being said additives.

6. Composition according to claim 1 characterized in that it comprises adrostenone, preferably 0.4 mm, androstenolo, preferably 0.7 mm, androsterone, preferably 0.2 mm, androstadienone, preferably 0.1 mm,, and copuline, preferably 0.1 mm, plus fragrances.

7. Method for the application and dispensation of synthetic human pheromones in clothing, underwear, jewels and like characterized in that it comprises the predisposition of a composition comprising at least three positive human substances, particularly androstenone, androstenediol, androsterone and copulins in form of a pressure or dissolution capsule, the positioning of at least one biocapsule in clothing, underwear, jewel and like and the technological biocapsule of said at least one biocapsule, in order to release the composition therein contained.

8. Use of a composition according to claim 1 in order to obtain the stimulation effect of VNO for the unconscious recognition of chemicals determining sexual attraction.

9. Clothing, underwear, jewel and like, comprising a composition as defined in claim 1.

10. Seamless capsule, wherein a composition according to claim 1 is located, which assumes a perfect shape like a bright pearl, said capsule allowing a dropping distribution.

11. Capsule according to claim 10 characterized in that the spreader thereof is constituted of a concentric double spreader, with an internal and external spreader, wherein the internal spreader allows the content to be leaked and the external spreader supplies the inside enclosed heated composition.
